(19) United States
(12) Patent Application Publication
(10) Pub. No.: US 2020/0373955 A1
(43) Pub. Date: Nov. 26, 2020

(54) RECEIVING DEVICE AND RECEIVING METHOD

(71) Applicant: OLYMPUS CORPORATION, Tokyo (JP)

(72) Inventors: Yusuke SUZUKI, Tokyo (JP); Toru MIYAZONO, Tokyo (JP)

(73) Assignee: OLYMPUS CORPORATION, Tokyo (JP)

(21) Appl. No.: 16/991,615

(22) Filed: Aug. 12, 2020

Related U.S. Application Data
(63) Continuation of application No. PCT/JP2018/030579, filed on Aug. 17, 2018.

(30) Foreign Application Priority Data
Mar. 8, 2018 (JP) 2018-042178

(51) Int. Cl. H04B 1/18; A61B 1/00; A61B 1/04

(52) U.S. Cl. CPC H04B 1/18; A61B 1/041; A61B 1/00016

(57) ABSTRACT

A receiving device includes: a receiving antenna configured to receive a wireless signal; a terminal circuit provided to the receiving antenna; a cable configured to transmit the wireless signal, one end of the cable being connected to the receiving antenna or the terminal circuit; a switch configured to switch between a first connection state where the one end of the cable is connected to the receiving antenna and a second connection state where the one end is connected to the terminal circuit; a measuring circuit configured to measures a first received strength of the wireless signal received by the receiving antenna and the cable in the first connection state and a second received strength of the wireless signal received by the cable in the second connection state; and a calculator configured to calculate, based on the first and second received strengths, a third received strength of the wireless signal.

This application is a continuation of PCT international application Ser. No. PCT/JP2018/030579, filed on Aug. 17, 2018 which designates the United States, incorporated herein by reference, and which claims the benefit of priority from Japanese Patent Applications No. 2018-042178, filed on Mar. 8, 2018, incorporated herein by reference.

### BACKGROUND

The present disclosure relates to a receiving device and a receiving method for receiving image data from outside.

In the field of endoscopes, capsule endoscopes have been developed (as seen in, for example, Japanese Patent Application Laid-open No. 2003-19111), the capsule endoscopes being body-insertable devices formed in sizes insertable in digestive tracts of subjects, such as patients. A capsule endoscope is a device having an imaging function and a wireless communication function inside a capsule casing; and after being swallowed from the mouth of a subject, the capsule endoscope acquires image data by sequentially capturing images of the interior of organs of the subject while moving in the digestive tract by peristaltic movement, and wirelessly transmits the acquired image data to a receiving device attached to the subject.

The receiving device sequentially receives wireless signals transmitted from the capsule endoscope via one or plural receiving antennas arranged distributedly over a surface of the body of the subject and causes the image data and received strength data of received radio waves to be sequentially recorded into a recording medium. The one or plural receiving antennas transmit the received wireless signals to the receiving device via a cable or cables. An image processing device fetches the image data and received strength data recorded in the recording medium and causes an image that has been subjected to predetermined image processing and a position of the capsule endoscope to be displayed on a display device, the position having been detected based the received strength data. A user, such as a medical doctor, observes the image and the position of the capsule endoscope relative to the subject, which have been displayed on the display device, and diagnose the subject.

### SUMMARY

According to one aspect of the present disclosure, there is provided a receiving device including: a receiving antenna configured to receive a wireless signal transmitted from a radio wave transmitting device; a terminal circuit provided to the receiving antenna; a cable configured to transmit the wireless signal, one end of the cable being connected to the receiving antenna or the terminal circuit; a switch configured to switch, in a time period in which the wireless signal is transmitted, between a first connection state where the one end of the cable is connected to the receiving antenna and a second connection state where the one end of the cable is connected to the terminal circuit; a measuring circuit configured to measures a first received strength of the wireless signal received by the receiving antenna and the cable in the first connection state and a second received strength of the wireless signal received by the cable in the second connection state; and a calculator configured to calculate, based on the first and second received strengths, a third received strength of the wireless signal received by the receiving antenna.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of a capsule endoscope system according to a first embodiment;

FIG. 2 is a block diagram illustrating a schematic configuration of the capsule endoscope system according to the first embodiment;

FIG. 3 is a diagram illustrating a configuration of main parts of the capsule endoscope system according to the first embodiment;

FIG. 4 is a timing chart for explanation of transmission of a wireless signal, switching by a switch, measured received strength, and corrected received strength, in the capsule endoscope system according to the first embodiment;

FIG. 5 is a timing chart for explanation of transmission of a wireless signal, switching by a switch, measured received strength, and corrected received strength, in a capsule endoscope system according to a modified example of the first embodiment;

FIG. 6 is a schematic diagram illustrating a schematic configuration of a capsule endoscope system according to a second embodiment;

FIG. 7 is a block diagram illustrating a schematic configuration of the capsule endoscope system according to the second embodiment;

FIG. 8 is a diagram illustrating a configuration of main parts of the capsule endoscope system according to the second embodiment;

FIG. 9 is a timing chart for explanation of transmission of a wireless signal, switching by switches, measured received strength, and corrected received strength, in the capsule endoscope system according to the second embodiment;

FIG. 10 is a block diagram illustrating a schematic configuration of a capsule endoscope system according to a third embodiment;

FIG. 11 is a diagram illustrating a configuration of main parts of the capsule endoscope system according to the third embodiment;

FIG. 12 is a timing chart for explanation of transmission of a wireless signal, switching by switches, measured received strength, and corrected received strength, in the capsule endoscope system according to the third embodiment; and

FIG. 13 is a timing chart for explanation of transmission of a wireless signal, switching by a switch, measured received strength, and corrected received strength, in a capsule endoscope system according to a fourth embodiment.

### DETAILED DESCRIPTION

Capsule endoscope systems each including a receiving device and using a medical capsule endoscope will be described below as embodiments according to the present disclosure. The same parts are assigned with the same reference signs, throughout the drawings.

### First Embodiment

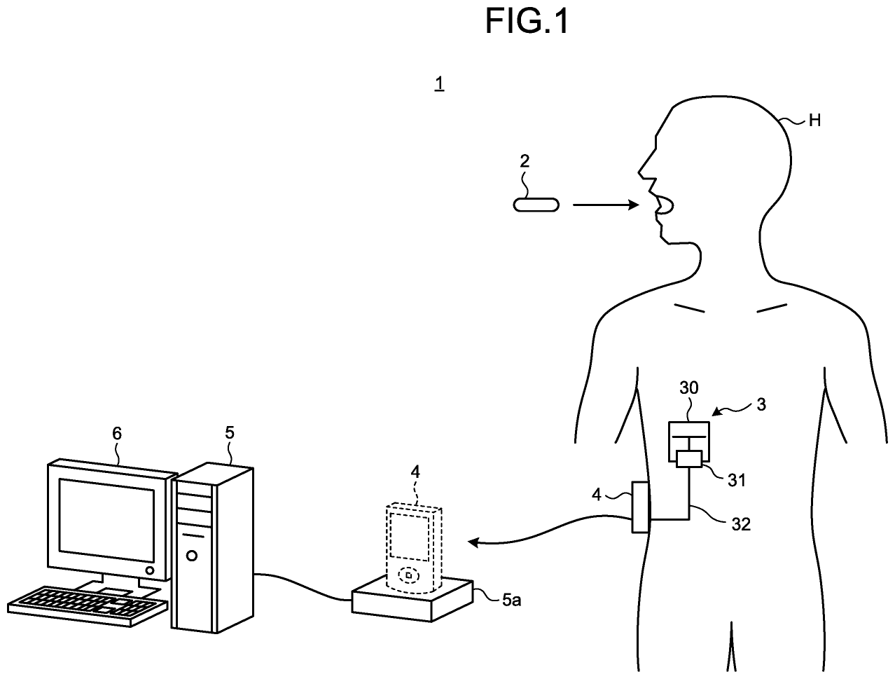

FIG. 1 is a schematic diagram illustrating a schematic configuration of a capsule endoscope system according to a first embodiment. As illustrated in FIG. 1, a capsule endoscope system 1 according to the first embodiment includes: a capsule endoscope 2 that is introduced into a subject H, generates image data by capturing an image of the interior of the subject H, superimposes the image data on a wireless signal, and transmits the image data superimposed on the wireless signal by using radio waves; a receiving device 4 that receives the wireless signal transmitted from the capsule endoscope 2 via a receiving antenna unit 3; and a processing device 5 that fetches the image data generated by the capsule endoscope 2 via a cradle 5a, processes the image data, and generates an image of the interior of the subject H. The image generated by the processing device 5 is, for example, output and displayed by the display device 6. In this specification, an image generated by the capsule endoscope 2 and in a state of having been converted into a format for transmission from the capsule endoscope 2 to the processing device 5 is called image data. The capsule endoscope 2 corresponds to a radio wave transmitting device.

After being swallowed by the subject H, the capsule endoscope 2 sequentially captures images of parts of the living body (the esophagus, the stomach, the small intestine, and the large intestine) at preset reference cycles (for example, at 0.5-second cycles) or cycles set as appropriate, while moving in the digestive tract of the subject H by peristaltic movement of the organs. The image data and related information acquired by this imaging operation are sequentially transmitted wirelessly to the receiving device 4.

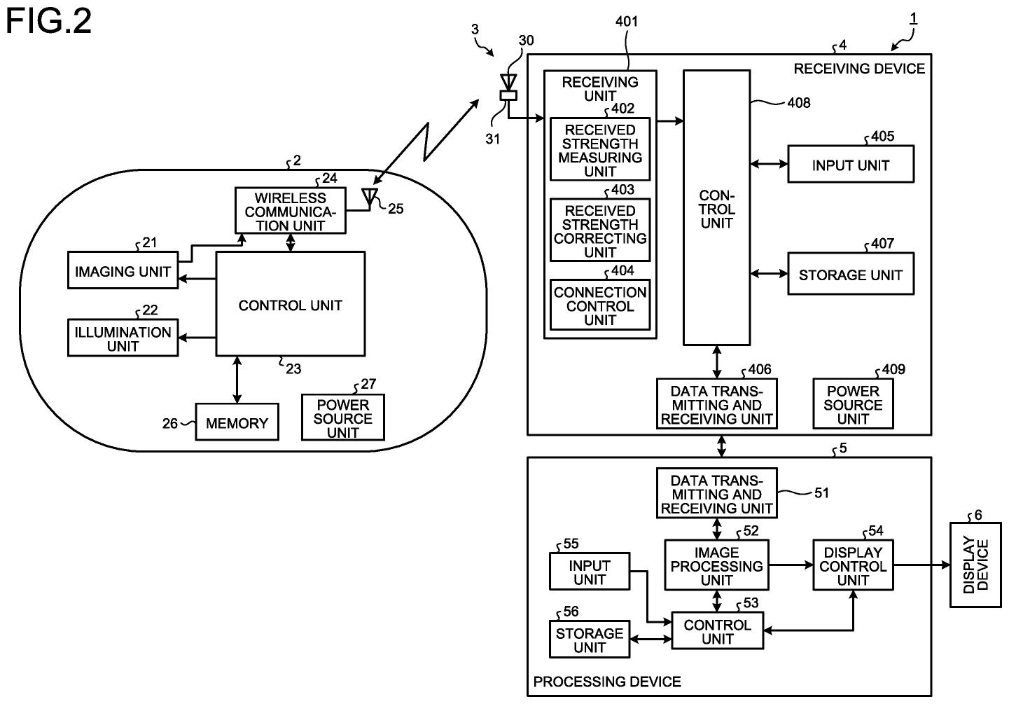

FIG. 2 is a block diagram illustrating a schematic configuration of the capsule endoscope system according to the first embodiment. The capsule endoscope 2 includes an imaging unit 21, an illumination unit 22, a control unit 23, a wireless communication unit 24, an antenna 25, a memory 26, and a power source unit 27. The capsule endoscope 2 is a device having the above described built-in components in a capsule-shaped casing having a size swallowable by the subject H.

The imaging unit 21 includes, for example: an imaging element that generates, from an optical image formed on a light receiving surface thereof, image data resulting from imaging of the interior of the subject H, and outputs the generated image data; and an optical system, such as an objective lens, which is placed on a light receiving surface side of the imaging element. The imaging element has plural pixels, each of which receives light from the subject H, and which are arranged in a matrix, and generates image data by photoelectrically converting the light received by the pixels. From the plural pixels arranged in a matrix, the imaging unit 21 reads pixel values per horizontal line and generates image data including plural sets of line data having a synchronization signal assigned to each horizontal line. The imaging unit 21 includes a charge coupled device (CCD) imaging element, or a complementary metal oxide semiconductor (CMOS) imaging element.

The illumination unit 22 includes, for example, a white light emitting diode (LED) that generates white light serving as illumination light. Instead of including a white LED, the illumination unit 22 may be configured to generate white light by combining light of plural LEDs or laser light sources that have different emission wavelength bands or may be configured using a xenon lamp or a halogen lamp.

The control unit 23 controls operation and processing of each component of the capsule endoscope 2. For example, when the imaging unit 21 performs imaging processing, the control unit 23 causes the imaging element to execute exposure processing and reading processing and causes the illumination unit 22 to emit illumination light according to the exposure timing of the imaging unit 21. Furthermore, the control unit 23 determines, from pixel values (luminances) of image data captured by the imaging unit 21, a light emission time period or a light emission amount of the illumination unit 22 for the next imaging, and causes the illumination unit 22 to emit illumination light over the determined light emission time period or for the determined light emission amount. The time period or amount of light emission by the illumination unit 22 is controlled by the control unit 23 based on the image data captured and thus each time imaging is performed, the light emission time period or light emission amount may change. The control unit 23 is formed using a general-purpose processor, such as a central processing unit (CPU), or a special-purpose processor, such as an arithmetic circuit that executes a specific function, like an application specific integrated circuit (ASIC).

The wireless communication unit 24 performs modulation processing on image data output from the imaging unit 21 and transmits the modulated image data to outside. The wireless communication unit 24 acquires digital image data by performing A/D conversion and predetermined signal processing on the image data output from the imaging unit 21, superimposes the digital image data, together with related information, on a wireless signal, and transmits the superimposed digital image data to outside, from the antenna 25. The related information includes identification information (for example, a serial number) that has been allocated for identification of the individuality of the capsule endoscope 2.

The memory 26 stores therein an execution program and a control program, for the control unit 23 to execute various types of operation, and parameters, such as thresholds. Furthermore, the memory 26 may temporarily store therein image data that have been signal-processed by the wireless communication unit 24. The memory 26 includes a random access memory (RAM) and a read only memory (ROM).

The power source unit 27 includes: a battery formed of, for example, a button cell; a power source circuit that supplies electric power to each unit; and a power source switch that switches the power source unit 27 between an on-state and an off-state; and the power source unit 27 supplies electric power to each unit in the capsule endoscope 2 after the power source switch has been turned on. The power source switch is formed of, for example, a reed switch that is switched between an on-state and an off-state by external magnetic force, and is switched to the on-state by application of magnetic force from outside to the capsule endoscope 2 before the capsule endoscope 2 is used (that is, before the capsule endoscope 2 is swallowed by the subject H).

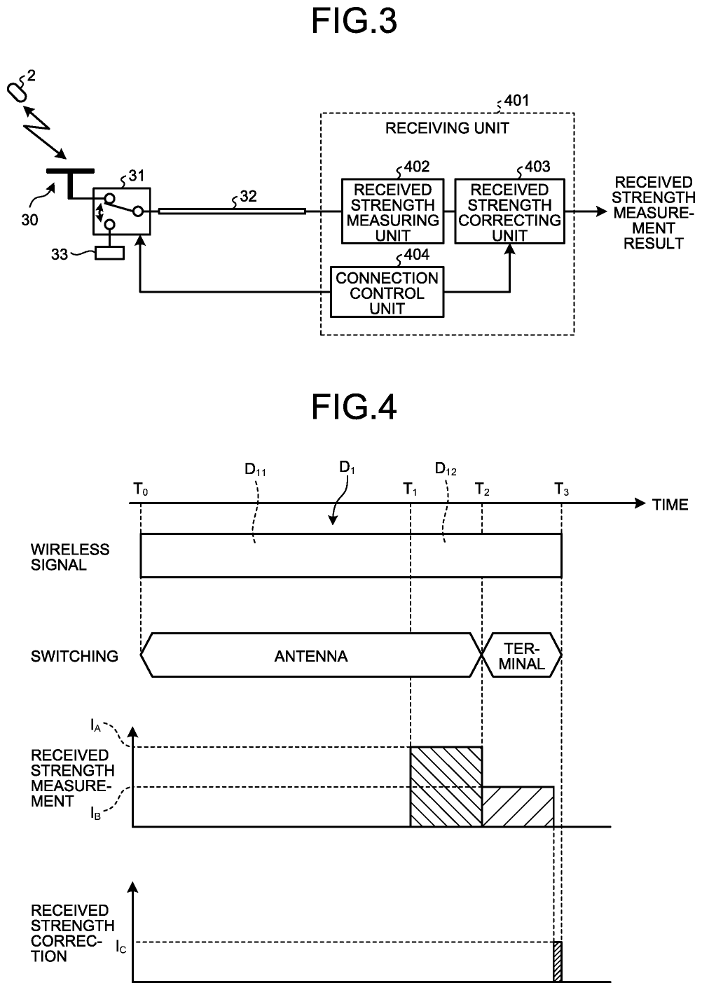

The receiving antenna unit 3 receives a wireless signal transmitted from the capsule endoscope 2. FIG. 3 is a diagram illustrating a configuration of main parts of the capsule endoscope system according to the first embodiment. The receiving antenna unit 3 includes a receiving antenna 30, a switch 31, a cable 32, and a terminal circuit 33.

The receiving antenna 30 includes an antenna element that receives a wireless signal transmitted from the capsule endoscope 2 and outputs the wireless signal to the cable 32. The antenna element is formed using a loop antenna or a dipole antenna.

Under control of the receiving device 4 (a connection control unit 404 described later), the switch 31 switches the connection destination of the cable 32 to either one of the receiving antenna 30 and the terminal circuit 33. The switch 31 is formed using a single-pole double-throw switch.

The cable 32 is formed using a signal line having one end connected to the switch 31 and the other end connected to the receiving device 4, and a tube that covers the signal line.

The terminal circuit 33 is a circuit formed using a terminating resistor. A characteristic impedance is set for the terminal circuit 33, according to a characteristic impedance of the cable 32.

The receiving device 4 includes a receiving unit 401, a received strength measuring unit 402, a received strength correcting unit 403, the connection control unit 404, an input unit 405, a data transmitting and receiving unit 406, a storage unit 407, a control unit 408, and a power source unit 409.

The receiving unit 401 receives a wireless signal wirelessly transmitted by the capsule endoscope 2. Specifically, the receiving unit 401 receives image data and related information that have been wirelessly transmitted from the capsule endoscope 2, via the receiving antenna unit 3. The receiving unit 401 has the received strength measuring unit 402, the received strength correcting unit 403, and the connection control unit 404. The receiving unit 401 performs predetermined signal processing, such as modulation processing, on the received image data. The receiving unit 401 is formed using: a general-purpose processor, such as a CPU; or a special-purpose processor, such as an arithmetic circuit that executes a specific function, like an ASIC.

The received strength measuring unit 402 measures a received strength, for example, a received signal strength indicator (RSSI), of a wireless signal received by the receiving antenna 30 and the cable 32. The receiving unit 401 is formed using: a general-purpose processor, such as a CPU; or a special-purpose processor, such as an arithmetic circuit that executes a specific function, like an ASIC.

The received strength correcting unit 403 corrects the received strength, for example, the RSSI, measured by the received strength measuring unit 402. The received strength correcting unit 403 calculates a difference between: the received strength of the wireless signal received by the receiving antenna 30 and the cable 32; and a received strength received by just the cable 32. The received strength correcting unit 403 determines this difference as a corrected received strength. This corrected received strength corresponds to a received strength of the wireless signal received by the receiving antenna 30. The received strength correcting unit 403 outputs the calculated corrected received strength as a received strength measurement result. The received strength correcting unit 403 may store the calculated corrected received strength and the image data received by the receiving unit 401 into the storage unit 407 in association with each other. The received strength correcting unit 403 is formed using: a general-purpose processor, such as a CPU; or a special-purpose processor, such as an arithmetic circuit that executes a specific function, like an ASIC. The received strength correcting unit 403 corresponds to a received strength calculating unit.

The connection control unit 404 causes the switch 31 to switch the connection destination of the cable 32 to either one of the receiving antenna 30 and the terminal circuit 33. The connection control unit 404 causes the switch 31 to change the connection destination of the cable 32 in synchronization with a timing for reception of a wireless signal from the capsule endoscope 2. The connection control unit 404 is formed using: a general-purpose processor, such as a CPU; or a special-purpose processor, such as an arithmetic circuit that executes a specific function, like an ASIC.

The input unit 405 is an input device that is used when a user inputs various types of setting information and instruction information to the receiving device 4. The input unit 405 is, for example, a switch or a button provided on an operation panel of the receiving device 4.

The data transmitting and receiving unit 406 transmits image data and related information that have been stored in the storage unit 407, to the processing device 5, when the data transmitting and receiving unit 406 is connected to the processing device 5 in a state where the data transmitting and receiving unit 406 is able to communicate with the processing device 5. The data transmitting and receiving unit 406 includes a communication interface, such as a LAN.

The storage unit 407 stores therein: a program for causing the receiving device 4 to operate and execute various functions; and image data acquired by the capsule endoscope 2. The storage unit 407 includes a RAM and a ROM.

The control unit 408 controls each component of the receiving device 4. The control unit 408 is formed using: a general-purpose processor, such as a CPU; or a special-purpose processor, such as an arithmetic circuit that executes a specific function, like an ASIC.

The power source unit 409 supplies electric power to each unit of the receiving device 4. The power source unit 409 is formed using a battery formed of an electric battery.

This receiving device 4 is attached to and carried by the subject H while imaging is being performed by the capsule endoscope 2, for example, while the capsule endoscope 2 is passing through the digestive tract after being swallowed by the subject H until the capsule endoscope 2 is excreted. The receiving device 4 stores image data received via the receiving antenna unit 3 during this imaging, into the storage unit 407.

After the imaging by the capsule endoscope 2 is ended, the receiving device 4 is removed from the subject H and set in the cradle 5a (as seen FIG. 1) connected to the processing device 5. As a result, the receiving device 4 is connected to the processing device 5 in a state of being communicable with the processing device 5 and transfers (downloads) the image data and related information stored in the storage unit 407, to the processing device 5.

The processing device 5 is formed using, for example, a work station including the display device 6, such as a liquid crystal display. The processing device 5 includes a data transmitting and receiving unit 51, an image processing unit 52, a control unit 53, a display control unit 54, an input unit 55, and a storage unit 56.

The data transmitting and receiving unit 51 is connected to the receiving device 4 via the cradle 5a and transmits and receives data to and from the receiving device 4. The data transmitting and receiving unit 51 includes a communication interface, such as a USB or a LAN.

By reading a predetermined program stored in a storage unit 58 described later, the image processing unit 52 executes predetermined image processing for generating an image corresponding to image data that have been input from the data transmitting and receiving unit 51 or image data that have been stored in the storage unit 58. The image processing unit 52 is implemented by a processor, such as a CPU or an ASIC.

By reading various programs stored in the storage unit 56, the control unit 53 executes, based on signals input via an input unit 57 and image data input from the data transmitting and receiving unit 51, transfer of instructions and data to the respective units forming the processing device 5 and integrally controls the overall operation of the processing device 5. The control unit 53 is implemented by: a general-purpose processor, such as a CPU; or a special-purpose processor, such as an arithmetic circuit that executes a specific functions, like an ASIC.

After performing predetermined processing, such as data thinning and/or gradation processing, on an image generated by the image processing unit 52, the predetermined processing corresponding to an image display range in the display device 6, the display control unit 54 causes the display device 6 to output and display the image acquired, together with information on a display target, such as a final score. The display control unit 54 includes, for example, a processor, such as a CPU or an ASIC.

The input unit 55 receives input of information or a command, which corresponds to a user's operation. The input unit 55 is implemented by an input device, such as, for example: a keyboard and a mouse; a touch panel; or various switches.

The storage unit 56 stores therein a program for causing the processing device 5 to operate and execute various functions, various kinds of information used during the execution of the program, image data and related information acquired from the receiving device 4, and endoscope images generated by the image processing unit 52. The storage unit 56 is implemented by: a semiconductor memory, such as a flash memory, a RAM, or a ROM; or a recording medium, such as an HDD, an MO, a CD-R, or a DVD-R, and a drive device that drives the recording medium.

Switching processing performed by the connection control unit 404, strength measurement processing performed by the received strength measuring unit 402, and correction processing performed by the received strength correcting unit 403 will be described next. FIG. 4 is a timing chart for explanation of transmission of a wireless signal, switching by a switch, measured received strength, and corrected received strength, in the capsule endoscope system according to the first embodiment.

The receiving unit 401 receives a wireless signal from the capsule endoscope 2. FIG. 4 illustrates a receiving time period of image data D₁ in wireless communication. The image data D₁ are formed of: a first region D₁₁ including pixel information related to pixels for generation of an image; and a second region D₁₂ including information that is other than the pixels of the first region D₁₁ and is related to horizontal/vertical synchronization for image generation. According to the first embodiment, the second region D₁₂ corresponds to a blanking time period. In FIG. 4, the image data D₁ corresponding to image generation data of one frame start being transmitted at a time T₀, data of the first region D₁₁ are transmitted from the time T₀ to a time T₁, and data of the second region D₁₂ are transmitted from the time T₁ to a time T₃. In the following description, transmission of data from the capsule endoscope 2 and reception of data at the receiving unit 401 are assumed to be performed substantially simultaneously, and the transmission time and the reception time are regarded as being the same.

At a first switching step, the connection control unit 404 controls the connection destination of the cable 32 to be the receiving antenna 30 in a time period from the time T₀ to a time T₂ in FIG. 4, the time period being in the time period in which the image data D₁ are received and being: the time period in which the data of the first region D₁₁ are received; and a part of the time period in which the data of the second region D₁₂ are received. Thereafter, at a second switching step, the connection control unit 404 controls the connection destination of the cable 32 to be the terminal circuit 33 in a time period from the time T₂ to the time T₃ in FIG. 4, the time period being in the time period in which the image data D₁ are received and being the remaining time period in which the data of the second region D₁₂ are received.

The receiving unit 401 receives, as data for image generation, the data of the first region D₁₁ of image data, in the time period in which the data of the first region D₁₁ are received, that is, the time period from the time T₀ to the time T₁, the time period being in the time period in which the image data D₁ are received. Thereafter, the receiving unit 401 receives, as first data for strength measurement, the data of the second region D₁₂ of the image data, in the part of the time period in which the data of the second region D₁₂ are received, that is, the time period from the time T₁ to the time T₂, the time period being in the time period in which the image data D₁ are received. The receiving unit 401 receives, as second data for strength measurement, the data of the second region D₁₂ of the image data, in the part of the time period in which the data of the second region D₁₂ are received, that is, the time period from the time T₂ to the time T₃, the part following the time T₂.

The time period in which the first data for strength measurement are received is a time period in which the receiving antenna 30 and the cable 32 are capable of receiving a wireless signal by control of the switch 31. At a measuring step, the received strength measuring unit 402 measures a received strength from the first data for strength measurement acquired in this time period. Therefore, a received signal strength indicator I_{A} measured from the first data for strength measurement includes received strengths of the wireless signal received by the receiving antenna 30 and the cable 32.

On the contrary, the time period in which the second data for strength measurement are received is a time period in which only the cable 32 is capable of receiving the wireless signal by control of the switch 31. At the measuring step, the received strength measuring unit 402 measures a received strength from the second data for strength measurement acquired in this time period. Therefore, a received signal strength indicator T_{B} measured from the second data for strength measurement includes a received strength of the wireless signal received by the cable 32.

Thereafter, at a calculating step, the received strength correcting unit 403 calculates a corrected received signal strength indicator I_{C} by calculating a difference (I_{A}−I_{B}) between the received signal strength indicator I_{A} of the wireless signal received by the receiving antenna 30 and the cable 32 and the received signal strength indicator I_{B} received by just the cable 32.

At the receiving unit 401, a received strength of the wireless signal transmitted from the capsule endoscope 2 and received by the receiving antenna 30 is calculated. The corrected received signal strength indicator I_{C} calculated by the received strength correcting unit 403 is a received strength of the wireless signal received just by the receiving antenna 30, the received strength not including the received strength of the wireless signal received by the cable 32. By using this corrected received strength, the receiving device 4 and the processing device 5 perform, for example, detection of a position of the capsule endoscope 2 and control of the imaging frame rate in the imaging processing by the capsule endoscope 2. The detection of a position of the capsule endoscope 2 includes detecting a position of the capsule endoscope 2 based on a corrected received strength and a position of the receiving antenna 30. A position of the capsule endoscope 2 may be detected using a known detecting method. The control of the imaging frame rate includes setting an imaging frame rate from an amount of change in the corrected received strength. For example, if the amount of change is large, the imaging frame rate is decreased, and if the amount of change is small, the imaging frame rate is increased. Furthermore, the position where the capsule endoscope 2 is passing in the subject H may be determined from the received strength, and the imaging frame rate may be controlled according to an organ presumed to be at that position. For example, if the capsule endoscope 2 is presumed to be passing the esophagus, a high imaging frame rate (of, for example, 20 fps to 60 fps) suitable for observation of the esophagus is set, and if the capsule endoscope 2 is presumed to have entered the stomach after passing the esophagus, a low imaging frame rate (of, for example, about 2 fps) is set.

According to the above described first embodiment, in a time period in which a wireless signal is received from the capsule endoscope 2, the connection destination of the cable 32 is controlled to be either one of the receiving antenna 30 and the terminal circuit 33, and a received strength of the wireless signal received by the receiving antenna 30 and the cable 32 and a received strength of the wireless signal received by just the cable 32 are acquired. Thereafter, at the receiving unit 401, based on the received strengths that have been received, the received strength correcting unit 403 calculates a received strength at just the receiving antenna 30. The first embodiment enables accurate measurement of the received strength of the wireless signal received by the receiving antenna 30.

According to the above description of the first embodiment, the received strength measuring unit 402 and the received strength correcting unit 403 calculate a received strength by using a digitized signal, but a received strength may be calculated by receiving an analog signal for strength measurement.

Furthermore, according to the above description of the first embodiment, the second region D₁₂ (blanking time period) is provided in the aftermost portion of the image data D₁, but the second region D₁₂ may be provided at the foremost portion or the central portion of the image data D₁. The connection control unit 404 may control the switch 31 according to the position of the second region D₁₂.

### Modified Example of First Embodiment

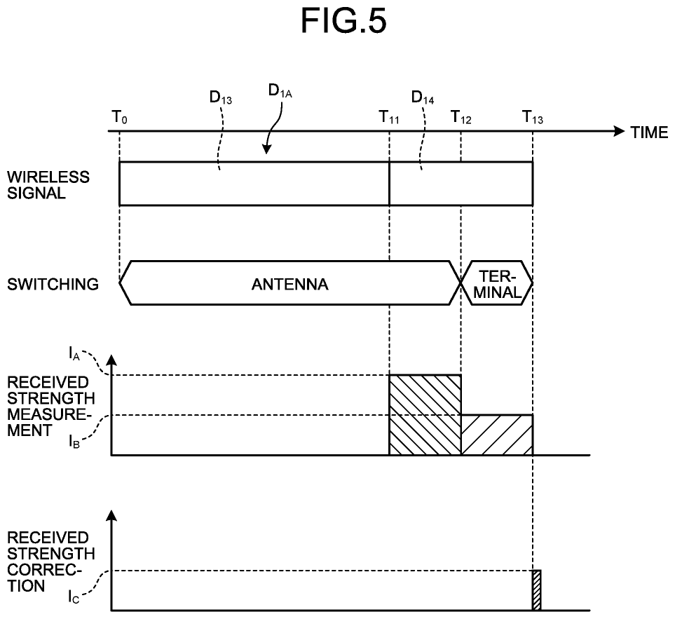

A modified example of the first embodiment will be described next. FIG. 5 is a timing chart for explanation of transmission of a wireless signal, switching by a switch, measured received strength, and corrected received strength, in a capsule endoscope system according to the modified example of the first embodiment. The capsule endoscope system 1 according to the modified example has the same configuration as the capsule endoscope 1 described above. In this modified example, the mode of transmission of a wireless signal is different from that of the first embodiment described above. Parts different from those of the first embodiment described above will be described below by reference to FIG. 5.

The receiving unit 401 receives a wireless signal from the capsule endoscope 2. Image data D_{1A} according to this modified example are formed of two sets of data.

Specifically, the image data D_{1A} are formed of: image generation data D₁₃ for generation of one frame of an image; and strength measurement data D₁₄. In FIG. 5, the image data D_{1A} start being transmitted at a time T₀, the image generation data D₁₃ are transmitted from the time T₀ to a time T₁₁, and the strength measurement data D₁₄ are transmitted thereafter from the time T₁₁ to a time T₁₃. The image generation data D₁₃ include a first region and a second region (a blanking time period) as described above.

The connection control unit 404 controls the connection destination of the cable 32 to be the receiving antenna 30 in a time period from the time T₀ to a time T₁₂ in FIG. 5, the time period being in the time period in which the image data D_{1A} are received and being: the time period in which the image generation data D₁₃ are received; and a part of the time period in which that strength measurement data D₁₄ are received. Thereafter, the connection control unit 404 controls the connection destination of the cable 32 to be the terminal circuit 33 in a time period from the time T₁₂ to the time T₁₃ in FIG. 5, the time period being in the time period in which the image data D_{1A} are received and being the remaining time period in which the strength measurement data D₁₄ are received.

The receiving unit 401 receives, as data for image generation, data received in the time period in which the image generation data D₁₃ are received, that is, the time period from the time T₀ to the time T₁₁, the time period being in the time period in which the image data D_{1A} are received. Thereafter, the receiving unit 401 receives, as first data for strength measurement, data received in a part of the time period in which the strength measurement data D₁₄ are received, that is, the time period from the time T₁₁ to the time T₁₂, the part being in the time period in which the image data D_{1A} are received. The receiving unit 401 receives, as second data for strength measurement, data received in a part of the time period in which the strength measurement data D₁₄ are received, that is, the time period from the time T₁₂ to the time T₁₃, the part following the time T₁₂.

The time period in which the first data for strength measurement are received is, similarly to the first embodiment, a time period in which the receiving antenna 30 and the cable 32 are capable of receiving a wireless signal, by control of the switch 31. Therefore, a received signal strength indicator I_{A} measured from the first data for strength measurement includes received strengths of the wireless signal received by the receiving antenna 30 and the cable 32.

On the contrary, the time period in which the second data for strength measurement are received is, similarly to the first embodiment, a time period in which only the cable 32 is capable of receiving the wireless signal, by control of the switch 31. Therefore, a received signal strength indicator I_{B} measured from the second data for strength measurement includes a received strength of the wireless signal received by the cable 32.

Thereafter, the received strength correcting unit 403 calculates a corrected received signal strength indicator I_{C} by calculating a difference (I_{A}−I_{S}) between the received signal strength indicator I_{A} of the wireless signal received by the receiving antenna 30 and the cable 32 and the received signal strength indicator I_{B} received by just the cable 32. When this corrected received signal strength indicator I_{C} has been calculated, similarly to the first embodiment, the receiving device 4 and the processing device 5 execute predetermined processing by using the corrected received signal strength indicator I_{C}.

The above described modified example has effects similar to the above described effects of the first embodiment.

### Second Embodiment

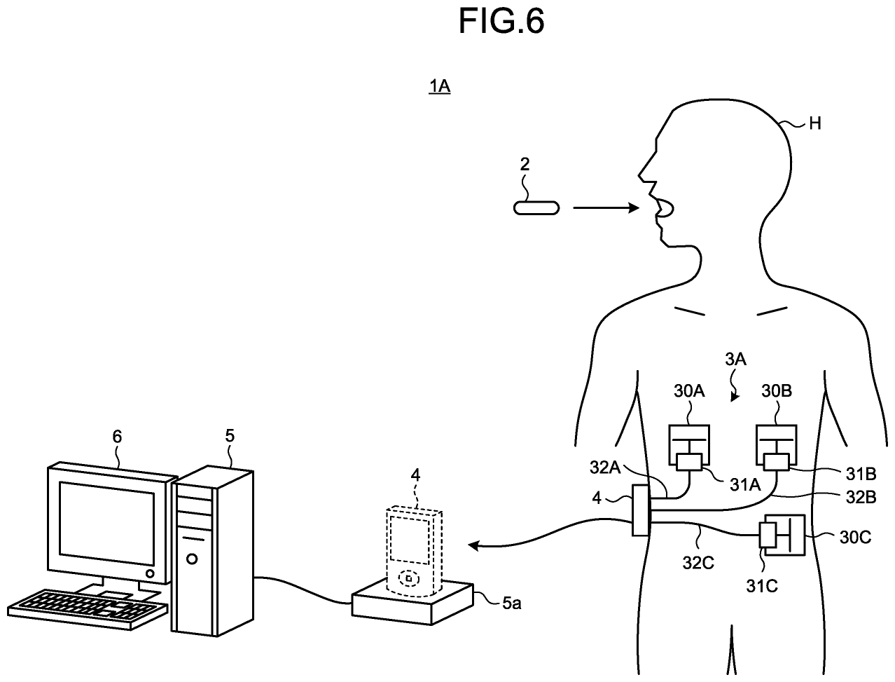

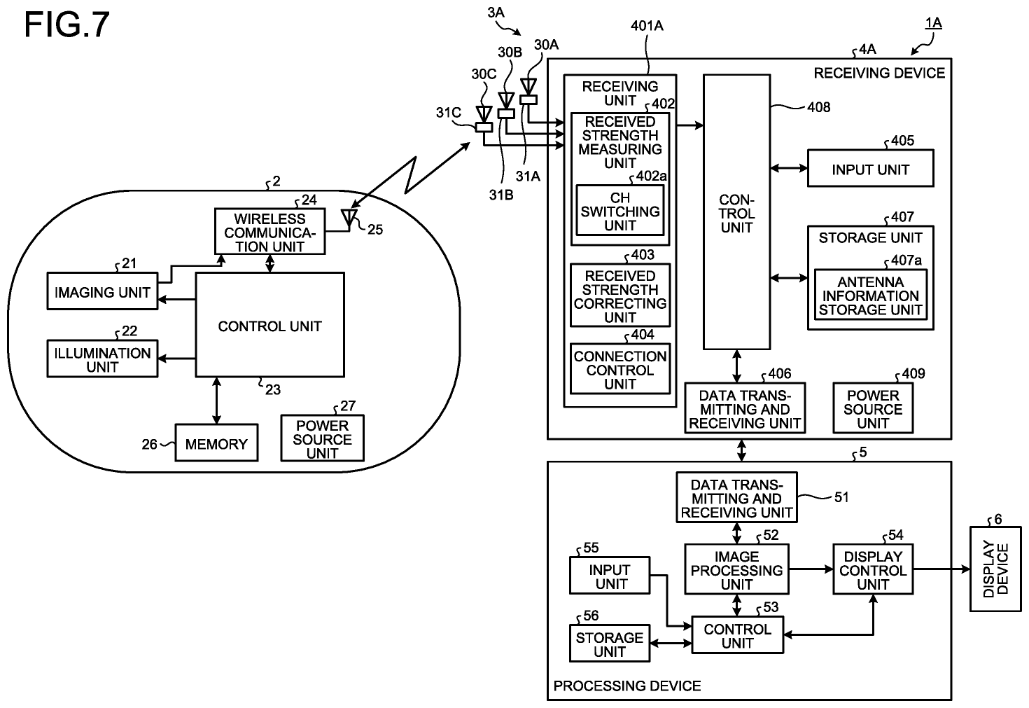

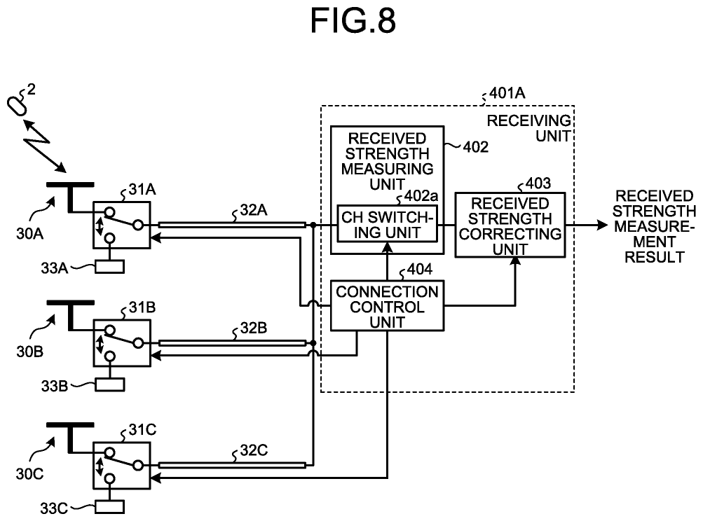

A second embodiment will be described next. FIG. 6 is a schematic diagram illustrating a schematic configuration of a capsule endoscope system according to a second embodiment. FIG. 7 is a block diagram illustrating a schematic configuration of the capsule endoscope system according to the second embodiment. FIG. 8 is a diagram illustrating a configuration of main parts of the capsule endoscope system according to the second embodiment.

A capsule endoscope system 1A according to the second embodiment is different from the capsule endoscope system 1 described above in that a receiving antenna unit 3A of the capsule endoscope system 1A includes plural receiving antennas (receiving antennas 30A to 30C) and further includes a receiving device 4A instead of the receiving device 4. The rest of the configuration of the capsule endoscope system 1A is the same as to that of the capsule endoscope system 1. Configurations and processing different from those according to the first embodiment described above will be described below by reference to FIG. 6 to FIG. 8.

The receiving antenna unit 3A includes the receiving antennas 30A, 30B, and 30C, switches 31A, 31B, and 31C, cables 32A, 32B, and 32C, and terminal circuits 33A, 33B, and 33C. Hereinafter, a transmission path formed using the receiving antenna 30A, the switch 31A, the cable 32A, and the terminal circuit 33A may be referred to as a first channel (CH1); a transmission path formed using the receiving antenna 30B, the switch 31B, the cable 32B, and the terminal circuit 33B may be referred to as a second channel (CH2); and a transmission path formed using the receiving antenna 30C, the switch 31C, the cable 32C, and the terminal circuit 33C may be referred to as a third channel (CH3).

The receiving antennas 30A, 30B, and 30C each include an antenna element that receives a wireless signal transmitted from the capsule endoscope 2 and outputs the wireless signal to the cable 32A, 32B, or 32C, respectively. The receiving antennas 30A, 30B, and 30C are able to be attached to a subject H independently from one another. The receiving antennas 30A, 30B, and 30C are attached to predetermined positions on the subject H. The receiving antennas 30A, 30B, and 30C are implemented using, for example, loop antennas or dipole antennas.

Under control of the receiving device 4 (the connection control unit 404), the switch 31A switches the connection destination of the cable 32A to either one of the receiving antenna 30A and the terminal circuit 33A. The switches 31B and 31C function similarly to the switch 31A. The switches 31A, 31B, and 31C are formed using single-pole double-throw switches.

The cable 32A is formed using a tube covering a signal line inserted therethrough, the signal line being connected to the switch 31A. The cables 32B and 32C are formed using, similarly to the cable 32A, signal lines respectively connected to the switches 31B and 31C and tubes covering these signal lines inserted therethrough. Each of the cables 32A, 32B, and 32C has a length corresponding to its attachment position on the subject H.

The terminal circuits 33A, 33B, and 33C are each formed using a terminating resistor. A characteristic impedance is set for each of the terminal circuits 33A, 33B, and 33C, according to characteristic impedances of cables connected thereto, the cables 32A, 32B, and 32C.

The receiving device 4A includes a receiving unit 401A, the received strength measuring unit 402, the received strength correcting unit 403, the connection control unit 404, the input unit 405, the data transmitting and receiving unit 406, the storage unit 407, the control unit 408, and the power source unit 409. Configurations of units other than the receiving unit 401A are the same as those of the capsule endoscope system 1. A configuration of the receiving unit 401A will be described below.

The receiving unit 401A receives a wireless signal wirelessly transmitted by the capsule endoscope 2. Specifically, the receiving unit 401A receives image data and related information that have been wirelessly transmitted from the capsule endoscope 2, via the receiving antenna unit 3A. The receiving unit 401A has the received strength measuring unit 402, the received strength correcting unit 403, and the connection control unit 404. The receiving unit 401A performs predetermined signal processing, such as modulation processing, on the received image data. The receiving unit 401A is formed using: a general-purpose processor, such as a CPU; or a special-purpose processor, such as an arithmetic circuit that executes a specific function, like an ASIC.

The received strength measuring unit 402 measures received strengths (RSSIs) of a wireless signal received by the receiving antennas 30A to 30C. The received strength measuring unit 402 has a CH switching unit 402a that selectively switches among received channel data. Under control of the connection control unit 404, the CH switching unit 402a switches among channels for receiving data.

The received strength correcting unit 403 corrects the received strength (the RSSI) at each receiving antenna measured by the received strength measuring unit 402. The received strength correcting unit 403 calculates, for example, a difference between a received strength of a wireless signal received by the receiving antenna 30A and the cable 32A and a received strength received by just the cable 32A. The received strength correcting unit 403 determines this difference as a corrected received strength at the receiving antenna 30A. This corrected received strength corresponds to a received strength of the wireless signal received by the receiving antenna 30A. The received strength correcting unit 403 outputs the calculated corrected received strength as a received strength measurement result.

The connection control unit 404 causes the switch 31A to switch the connection destination of the cable 32A to either one of the receiving antenna 30A and the terminal circuit 33A. The connection control unit 404 causes the switches 31B and 31C to switch between connection destinations of the cables 32B and 32C, similarly. The connection control unit 404 causes each switch to change the connection destination of the cable in synchronization with a timing for transmission of a wireless signal from the capsule endoscope 2.

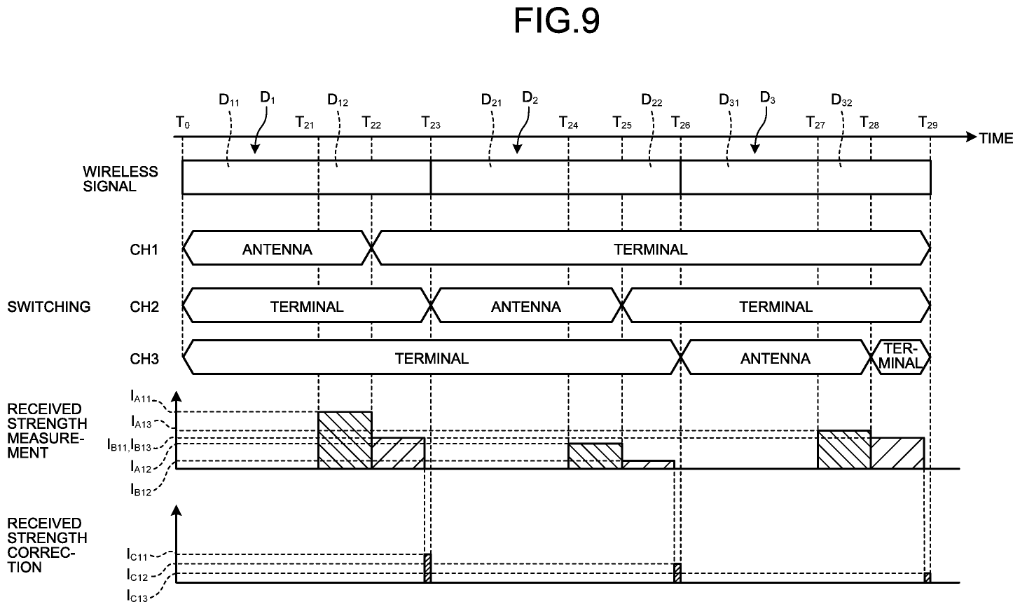

Switching processing performed by the connection control unit 404, strength measurement processing performed by the received strength measuring unit 402, and correction processing performed by the received strength correcting unit 403, according to the second embodiment, will be described next. FIG. 9 is a timing chart for explanation of transmission of a wireless signal, switching by switches, measured received strength, and corrected received strength, in the capsule endoscope system according to the second embodiment.

The receiving unit 401A receives a wireless signal from the capsule endoscope 2. Similarly to the image data D₁, image data D₂ illustrated in FIG. 9 are formed of a first region D₂₁ and a second region (blanking time period) D₂₂. Similarly, image data D₃ are formed of a first region D₃₁ and a second region (blanking time period) D₃₂. In FIG. 9, the image data D₁ start being transmitted at the time T₀, data of the first region D₁₁ are transmitted from the time T₀ to a time T₂₁, and data of the second region D₁₂ are transmitted from the time T₂₁ to a time T₂₃. Subsequently to the image data D₁, the image data D₂ start being transmitted at the time T₂₃, data in the first region D₂₁ are transmitted from the time T₂₃ to a time T₂₄, and data of the second region D₂₂ are transmitted from the time T₂₄ to a time T₂₆. Subsequently to the image data D₂, the image data D₃ start being transmitted at the time T₂₆, data in the first region D₃₁ are transmitted from the time T₂₆ to a time T₂₇, and data of the second region D₃₂ are transmitted from the time T₂₇ to a time T₂₉. According to this second embodiment, image data are transmitted in time periods shifted from one another among the channels, but transmitting and receiving time periods of image data are very short (for example, a few milliseconds) and thus the image data may be assumed to be received substantially simultaneously.

The connection control unit 404 controls the connection destination of the cable 32A to be the receiving antenna 30A in a time period from the time T₀ to a time T₂₂ in FIG. 9, the time period being in the time period in which the image data D₁ are received and being: the time period in which the data of the first region D₁₁ are received; and a part of the time period in which the data in the second region D₁₂ are received. Thereafter, the connection control unit 404 controls the connection destination of the cable 32A to be the terminal circuit 33A in a time period from the time T₂₂ to the time T₂₃ in FIG. 9, the time period being in the time period in which the image data D₁ are received and being the remaining time period in which the data of the second region D₁₂ are received. In this remaining time period, for the second and third channels, the connection destinations of the cables 32B and 32C have been controlled to be the terminal circuits 33B and 33C.

The connection control unit 404 controls the connection destination of the cable 32B to be the receiving antenna 30B in a time period from the time T₂₃ to a time T₂₅ in FIG. 9, the time period being in the time period in which the image data D₂ are received and being: the time period in which the data of the first region D₂₁ are received; and a part of the time period in which the data of the second region D₂₂ are received. Thereafter, the connection control unit 404 controls the connection destination of the cable 32B to be the terminal circuit 33B in a time period from the time T₂₅ to the time T₂₆ in FIG. 9, the time period being in the time period in which the image data D₂ are received and being the remaining time period in which the data of the second region D₂₂ are received. In this remaining time period, for the first and third channels, the connection destinations of the cables 32A and 32C have been controlled to be the terminal circuits 33A and 33C.

The connection control unit 404 controls the connection destination of the cable 32B to be the receiving antenna 30C in a time period from the time T₂₆ to a time T₂₈ in FIG. 9, the time period being in the time period in which the image data D₃ are received and being: the time period in which the data of the first region D₃₁ are received; and a part of the time period in which the data of the second region D₃₂ are received. Thereafter, the connection control unit 404 controls the connection destination of the cable 32C to be the terminal circuit 33C in a time period from the time T₂₈ to the time T₂₉ in FIG. 9, the time period being in the time period in which the image data D₃ are received and being the remaining time period in which the data of the second region D₃₂ are received. In this remaining time period, for the first and second channels, the connection destinations of the cables 32A and 32B have been controlled to be the terminal circuits 33A and 33B.

In the receiving unit 401A, the CH switching unit 402a causes the receiving unit 401A to receive the data of the first channel between the time T₀ and the time T₂₃. The receiving unit 401A receives, as data for image generation, the data of the first region D₁₁ of image data, in the time period in which the data of the first region D₁₁ are received, that is, the time period from the time T₀ to the time T₂₁, the time period being in the time period in which the image data D₁ are received. Thereafter, the receiving unit 401A receives, as first data for strength measurement, the data of the second region D₁₂ of the image data, in the part of the time period in which the data of the second region D₁₂ are received, that is, the time period from the time T₂₁ to the time T₂₂, the part being in the time period in which the image data D₁ are received. The receiving unit 401A receives, as second data for strength measurement, the data of the second region D₁₂ of the image data, in a part of the time period in which the data of the second region D₁₂ are received, that is, the time period from the time T₂₂ to the time T₂₃, the part following the time T₂₂.

Between the time T₂₃ to the time T₂₆, the CH switching unit 402a causes the receiving unit 401A to receive the data of the second channel. The receiving unit 401A receives, as data for image generation, the data of the first region D₂₁ of the image data, in the time period in which the data of the first region D₂₁ are received, that is, the time period from the time T₂₃ to the time T₂₄, the time period being in the time period in which the image data D₂ are received. Thereafter, the receiving unit 401A receives, as first data for strength measurement, the data of the second region D₂₂ of the image data, in the part of the time period in which the data of the second region D₂₂ are received, that is, the time period from the time T₂₄ to the time T₂₅, the part being in the time period in which the image data D₂ are received. The receiving unit 401A receives, as second data for strength measurement, the data of the second region D₂₂ of the image data, in the part of the time period in which the data of the second region D₂₂ are received, that is, the time period from the time T₂₅ to the time T₂₆, the part following the time T₂₅.

Between the time T₂₆ to the time T₂₉, the CH switching unit 402a causes the receiving unit 401A to receive the data of the third channel. The receiving unit 401A receives, as data for image generation, the data of the first region D₃₁ of the image data, in the time period in which the data of the first region D₃₁ are received, that is, the time period from the time T₂₆ to the time T₂₇, the time period being in the time period in which the image data D₃ are received. Thereafter, the receiving unit 401A receives, as first data for strength measurement, the data of the second region D₃₂ of the image data, in the part of the time period in which the data of the second region D₃₂ are received, that is, the time period from the time T₂₇ to the time T₂₈, the part being in the time period in which the image data D₃ are received. The receiving unit 401A receives, as second data for strength measurement, the data of the second region D₃₂ of the image data, in the part of the time period in which the data of the second region D₃₂ are received, that is, the time period from the time T₂₈ to the time T₂₉, the part following the time T₂₈.

The time periods in which the first data for strength measurement are received are time periods in which the receiving antennas and cables (cables 32A to 32C) are capable of receiving a wireless signal by control of the switches 31A to 31C. Therefore, received signal strength indicators Iₘ to Iₘ measured from the first data for strength measurement each include received strengths of the wireless signal received by the receiving antenna and the cable.

On the contrary, the time periods in which the second data for strength measurement are received are time periods in which only the cables (cables 32A to 32C) are capable of receiving a wireless signal by control of the switches 31A to 31C. Therefore, received signal strength indicators I_{B11} to I_{B13} measured from the second data for strength measurement each include a received strength of the wireless signal received by the cable.

Thereafter, the received strength correcting unit 403 calculates corrected received signal strength indicators T_{C11} to I_{C13} by calculating differences (for example, “I_{A11}−I_{B11}”) between the received strengths (for example, I_{A11}) of the wireless signal received by the receiving antennas and cables and the received strengths (for example, I_{B11}) received by just the cables.

The corrected received signal strength indicators I_{C11} to I_{C13} calculated as described above are received strengths of the wireless signal received by just the receiving antennas 30A to 30C, the received strengths not including the received strengths of the wireless signal received by the cables 32A to 32C. By using these corrected received strengths, the receiving device 4 and the processing device 5 perform, for example, detection of positions of the capsule endoscope 2 and control of the frame rate in the imaging processing of the capsule endoscope 2. According to the second embodiment, the positions of the capsule endoscope 2 are detected using the received strengths at the plural receiving antennas attached at the predetermined positions, and the positions are thus able to be detected more accurately, as compared to the first embodiment.

According to the second embodiment described above, the connection destination of a cable (the cables 32A to 32C) is controlled to be either one of a receiving antenna (the receiving antennas 30A to 30C) and a terminal circuit (the terminal circuits 33A to 33C) in a time period in which a wireless signal is received from the capsule endoscope 2, and a received strength of the wireless signal received by the receiving antenna and cable and a received strength of the wireless signal received by just the cable are acquired. Thereafter, at the receiving unit 401A, based on the received strengths that have been received, the received strength correcting unit 403 calculates a received strength at just the receiving antenna. This second embodiment enables accurate measurement of received strengths of a wireless signal received by the receiving antennas 30A to 30C.

According to the above description of the second embodiment, the processing from the reception of image data to the signal processing in the channels is continuous without any intervals between the channels, but intervals may be provided between the channels.

Like the modified example (as seen in FIG. 5) of the first embodiment, the second embodiment described above is also applicable to a case where a wireless signal includes image data and strength measurement data. In this case, a received strength is measured in a time period in which the strength measurement data are received.

### Third Embodiment

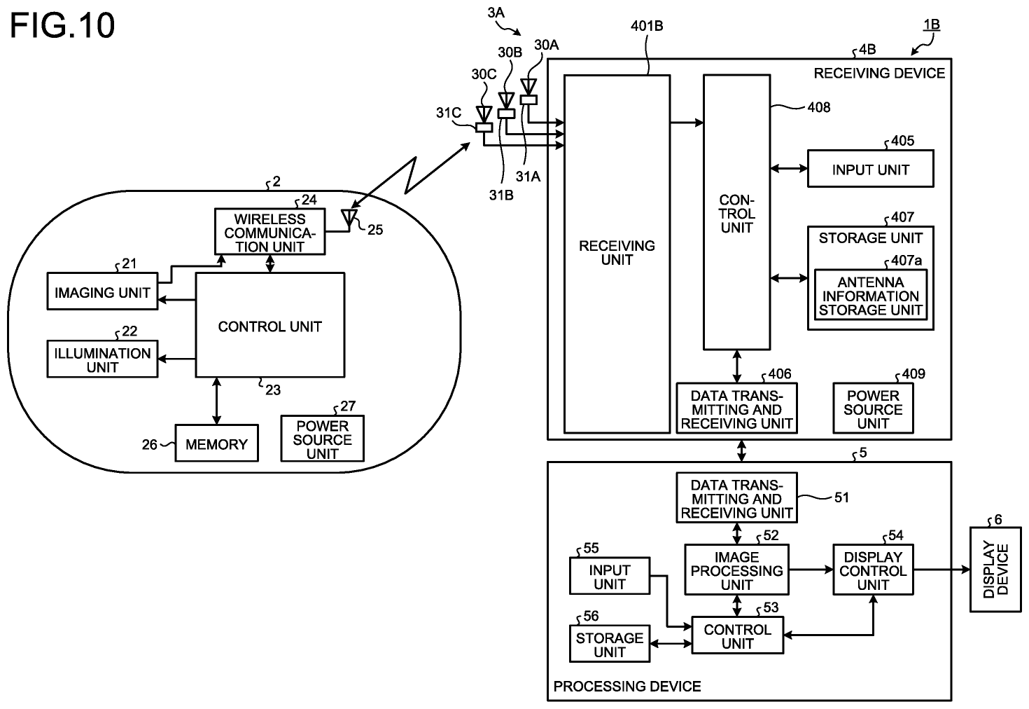

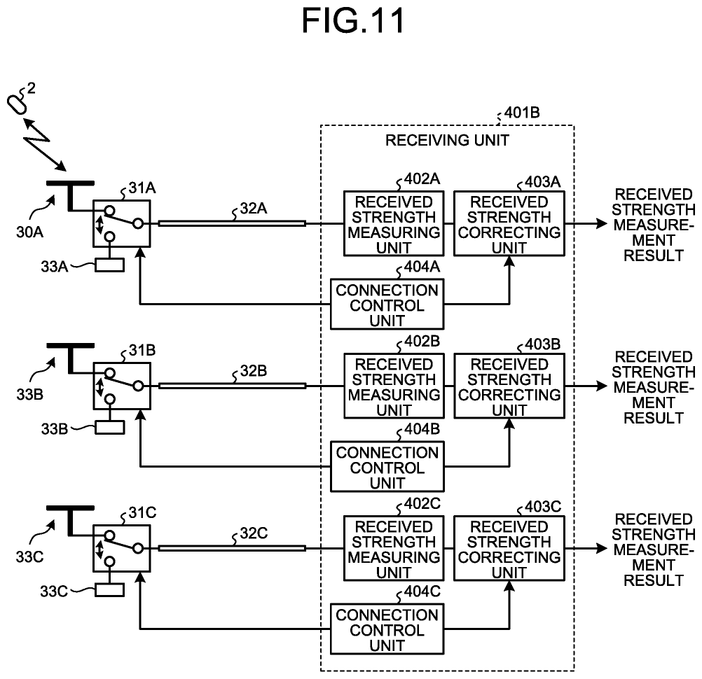

A third embodiment will be described next. FIG. 10 is a block diagram illustrating a schematic configuration of a capsule endoscope system according to the third embodiment. FIG. 11 is a diagram illustrating a configuration of main parts of the capsule endoscope system according to the third embodiment.

A capsule endoscope system 1B according to the third embodiment is different from the capsule endoscope system 1A described above in that the capsule endoscope system 1B includes a receiving device 4B instead of the receiving device 4A. The rest of the configuration of the capsule endoscope system 1B is the same as to that of the capsule endoscope system 1A. Configurations and processing different from those according to the second embodiment described above will be described below by reference to FIG. 10 and FIG. 11.

The receiving device 4B includes a receiving unit 401B, a received strength measuring unit 402, a received strength correcting unit 403, a connection control unit 404, the input unit 405, the data transmitting and receiving unit 406, the storage unit 407, the control unit 408, and the power source unit 409. Configurations of the units other than the receiving unit 401B are the same as those of the capsule endoscope systems 1 and 1A. A configuration of the receiving unit 401B will be described below.

The receiving unit 401B receives a wireless signal wirelessly transmitted by the capsule endoscope 2. Specifically, the receiving unit 401B receives image data and related information that have been wirelessly transmitted from the capsule endoscope 2, via the receiving antenna unit 3A. The receiving unit 401B has the received strength measuring unit 402, the received strength correcting unit 403, and the connection control unit 404. The receiving unit 401B performs predetermined signal processing, such as modulation processing, on the received image data. The receiving unit 401B is formed using: a general-purpose processor, such as a CPU; or a special-purpose processor, such as an arithmetic circuit that executes a specific function, like an ASIC.

The receiving unit 401B has, for each channel, a received strength measuring unit, a received strength correcting unit, and a connection control unit.

Specifically, the receiving unit 401B has: a received strength measuring unit 402A, a received strength correcting unit 403A, and a connection control unit 404A that are related to processing of data received from a first channel; a received strength measuring unit 402B, a received strength correcting unit 403B, and a connection control unit 404B that are related to processing of data received from a second channel; and a received strength measuring unit 402C, a received strength correcting unit 403C, and a connection control unit 404C that are related to processing of data received from a third channel.

The received strength measuring unit 402A measures a received strength (an RSSI) of a wireless signal received by the receiving antenna 30A.

The received strength measuring unit 402B measures a received strength (an RSSI) of a wireless signal received by the receiving antenna 30B.

The received strength measuring unit 402C measures a received strength (an RSSI) of a wireless signal received by the receiving antenna 30C.

The received strength correcting unit 403A corrects the received strength (the RSSI) at the receiving antenna 30A measured by the received strength measuring unit 402A. The received strength correcting unit 403A calculates a difference between a received strength of a wireless signal received by the receiving antenna 30A and the cable 32A and a received strength received by just the cable 32A. The received strength correcting unit 403A determines this difference as a corrected received strength at the receiving antenna 30A. This corrected received strength corresponds to a received strength of the wireless signal received by the receiving antenna 30A. The received strength correcting unit 403A outputs the calculated corrected received strength as a received strength measurement result. Similarly to the received strength correcting unit 403A, the received strength correcting units 403B and 403C calculate corrected received strengths of the receiving antennas 30B and 30C.

In synchronization with a timing for transmission of a wireless signal from the capsule endoscope 2, the connection control unit 404A causes the switch 31A to switch the connection destination of the cable 32A to either one of the receiving antenna 30A and the terminal circuit 33A. Similarly to the connection control unit 404A, the connection control units 404B and 404C cause the switches 31B and 31C to change the connection destinations of the cables 32B and 32C. The connection control units 404A to 404C perform switching of the connection states parallelly to one another.

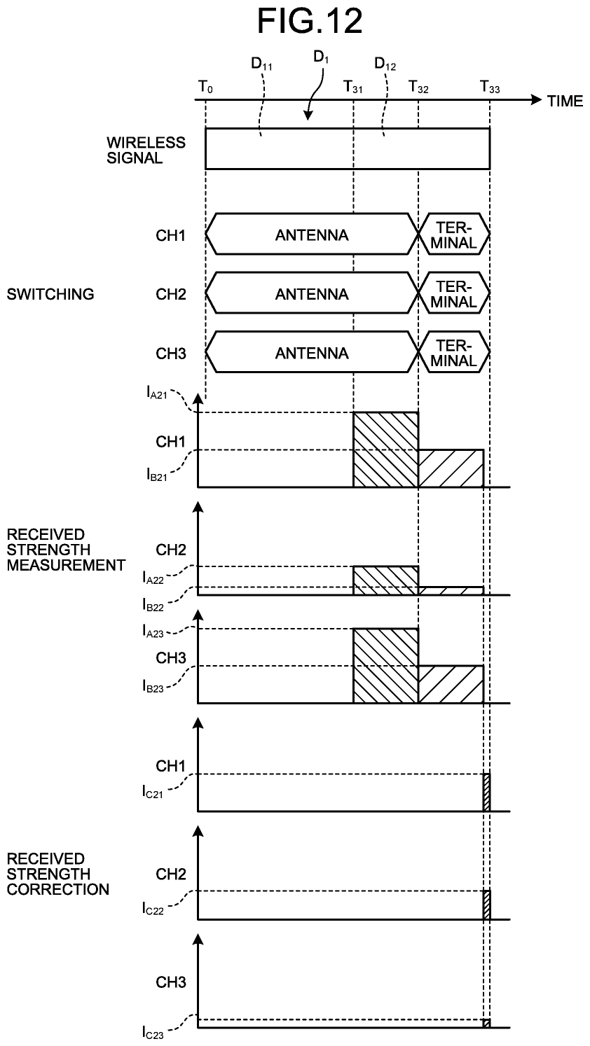

Switching processing performed by the connection control unit 404, strength measurement processing performed by the received strength measuring unit 402, and correction processing performed by the received strength correcting unit 403, according to the third embodiment, will be described next. FIG. 12 is a timing chart for explanation of transmission of a wireless signal, switching by switches, measured received strength, and corrected received strength, in the capsule endoscope system according to the third embodiment.

A receiving unit 401B receives a wireless signal from the capsule endoscope 2. In FIG. 12, image data D₁ start being transmitted at a time T₀, data of a first region D₁₁ are transmitted from the time T₀ to a time T₃₁, and data of a second region D₁₂ are transmitted from the time T₃₁ to a time T₃₃. According to this third embodiment, the channels receive the image data D₁ simultaneously.

The connection control unit 404A controls the connection destination of the cable 32A to be the receiving antenna 30A in a time period from the time T₀ to a time T₃₂ in FIG. 12, the time period being in the time period in which the image data D₁ are received and being: the time period in which the data of the first region D₁₁ are received; and a part of the time period in which the data of the second region D₁₂ are received. Thereafter, the connection control unit 404A controls the connection destination of the cable 32A to be the terminal circuit 33A in a time period from the time T₃₂ to the time T₃₃ in FIG. 12, the time period being in the time period in which the image data D₁ are received and being the remaining time period in which the data of the second region D₁₂ are received.

Similarly to the connection control unit 404A, the connection control unit 404B controls the connection destination of the cable 32B to be the receiving antenna 30B in the time period from the time T₀ to the time T₃₂. Thereafter, the connection control unit 404B controls the connection destination of the cable 32B to be the terminal circuit 33B in the time period from the time T₃₂ to the time T₃₃. Similarly, the connection control unit 404C controls the connection destination of the cable 32C to be the receiving antenna 30C in the time period from the time T₀ to the time T₃₂. Thereafter, the connection control unit 404C controls the connection destination of the cable 32C to be the terminal circuit 33C in the time period from the time T₃₂ to the time T₃₃.

The receiving unit 401B receives, as data for image generation, the data of the first region D₁₁ of image data, in the time period in which the data of the first region D₁₁ are received, that is, the time period from the time T₀ to the time T₃₁, the time period being in the time period in which the image data D₁ are received. Thereafter, the receiving unit 401B receives, as first data for strength measurement, the data of the second region D₁₂ of the image data, in the part of the time period in which the data of the second region D₁₂ are received, that is, the time period from the time T₃₁ to the time T₃₂, the part being in the time period in which the image data D₁ are received. The receiving unit 401B receives, as second data for strength measurement, the data of the second region D₁₂ of the image data, in the part of the time period in which the data of the second region D₁₂ are received, that is, the time period from the time T₃₂ to the time T₃₃, the part following the time T₃₂. As a result, the receiving unit 401B receives the data for image generation, the first data for strength measurement, and the second data for strength measurement, in each channel.

The received strength correcting units 403A to 403C each calculate a corrected received signal strength indicator I_{C} (I_{C21} to I_{C23}) by calculating a difference (for example, “I_{A21}−I_{B21}”) between a received signal strength indicator I_{A} (I_{A21} to I_{A23}) of a wireless signal received by a receiving antenna and a cable and a received signal strength indicator I_{B} (I_{B21} to I_{B23}) received at just the cable.

The corrected received signal strength indicator I_{C} calculated as described above is the received strength of the wireless signal received by just the receiving antenna 30A, 30B, or 30C, the received strength not including the received strength of the wireless signal received by the cable 32A, 32B, or 32C. By using these corrected received strengths, the receiving device 4B and the processing device 5 perform, for example, detection of positions of the capsule endoscope 2 and control of the frame rate in the imaging processing of the capsule endoscope 2.

According to the third embodiment described above, the connection destination of a cable (the cables 32A to 32C) is controlled to be either one of a receiving antenna (the receiving antennas 30A to 30C) and a terminal circuit (the terminal circuits 33A to 33C) in a time period in which a wireless signal is received from the capsule endoscope 2, and a received strength of the wireless signal received by the receiving antenna and cable and a received strength of the wireless signal received by just the cable are acquired. Thereafter, at the receiving unit 401B, based on the received strengths that have been received, the received strength correcting units 403A to 403C calculate received strengths of just the receiving antennas. This third embodiment enables accurate measurement of received strengths of a wireless signal received by the receiving antennas 30A to 30C. Furthermore, according to the third embodiment described above, the channels receive image data at the same time and thus electric power consumed by the capsule endoscope 2 is able to be reduced.

According to the above description of the third embodiment, each channel has one set of the received strength measuring unit, the received strength correcting unit, and the connection control unit, but for example, channel: (received strength measuring unit, received strength correcting unit, and connection control unit) may be M:N (where M>N).

Furthermore, according to the above description of the third embodiment, image data are simultaneously received by the channels, but the received times may differ among the channels.

### Fourth Embodiment

A fourth embodiment will be described next. A capsule endoscope system according to this fourth embodiment is similar to the capsule endoscope system 1 described above.

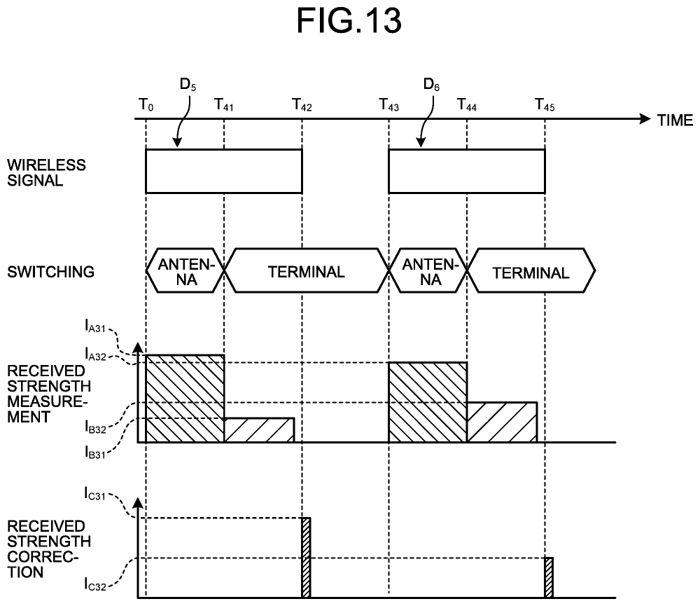

According to this fourth embodiment, a capsule endoscope 2 stores data for image generation into a memory 26 without wireless transmitting the data, and wireless transmits only data for strength measurement. A receiving device 4 may be formed without the data transmitting and receiving unit 406. Processing different from that according to the first embodiment described above will be described below by reference to FIG. 13.

FIG. 13 is a timing chart for explanation of transmission of a wireless signal, switching by a switch, measured received strength, and corrected received strength, in the capsule endoscope system according to the fourth embodiment.

The receiving unit 401 receives a wireless signal from the capsule endoscope 2. FIG. 13 illustrates receiving time periods for strength measurement data D₅ and D₆ in wireless communication. In FIG. 13, transmission of the strength measurement data D₅ is started at a time T₀ and completed at or before a time T₄₂. Transmission of the strength measurement data D₆ is started at a time T₄₃ at a predetermined interval from the time T₄₂ and completed at or before a time T₄₅. According to this fourth embodiment, the strength measurement data are transmitted intermittently.

The connection control unit 404 controls the connection destination of the cable 32 to be the receiving antenna 30 in a time period from the time T₀ to a time T₄₁ in FIG. 13, the time period being a part of the time period in which the strength measurement data D₅ are transmitted. Thereafter, the connection control unit 404 controls the connection destination of the cable 32 to be the terminal circuit 33 in a time period from the time T₄₁ to the time T₄₂ in FIG. 13, the time period being the remaining time period of the time period in which the strength measurement data D₅ are received.

Furthermore, the connection control unit 404 controls the connection destination of the cable 32 to be the receiving antenna 30 in a time period from the time T₄₃ to a time T₄₄ in FIG. 13, the time period being a part of the time period in which the strength measurement data D₆ are received. Thereafter, the connection control unit 404 controls the connection destination of the cable 32 to be the terminal circuit 33 in a time period from the time T₄₄ to the time T₄₅ in FIG. 13, the time period being the remaining time period of the time period in which the strength measurement data D₆ are received.

The receiving unit 401 receives, as first data for strength measurement in the strength measurement data D₅, the data received in the time period from the time T₀ to the time T₄₁. The receiving unit 401 receives, as second data for strength measurement in the strength measurement data D₅, the data received in the time period, which follows the time T₄₁ and in which data are received, that is, the time period from the time T₄₁ to the time T₄₂.

Furthermore, the receiving unit 401 receives, as first data for strength measurement in the strength measurement data D₆, data received in the time period from the time T₄₃ to the time T₄₄. The receiving unit 401 receives, as second data for strength measurement in the strength measurement data D₆, data received in the time period, which follows the time T₄₄ and in which data are received, that is, the time period from the time T₄₄ to the time T₄₅.

The received strength correcting unit 403 calculates a corrected received signal strength indicator I_{C} (I_{C31} or I_{C32}) by calculating a difference (for example, “I_{A31}−I_{B31}”) between a received signal strength indicator I_{A} (I_{A31} or I_{A32}) of a wireless signal received by a receiving antenna and a cable and a received signal strength indicator I_{B} (I_{B31} or I_{B32}) received by just the cable.

The calculated corrected received signal strength indicator I_{C} is a received strength of the wireless signal received by just the receiving antenna 30, the received strength not including the received strength of the wireless signal received by the cable 32. By using these corrected received strengths, the receiving device 4 and the processing device 5 perform, for example, detection of positions of the capsule endoscope 2 and control of the frame rate in the imaging processing of the capsule endoscope 2. Furthermore, image data generated by the capsule endoscope 2 are acquired from the memory 26 after the capsule endoscope 2 has been excreted from the subject H.

According to the fourth embodiment described above, similarly to the first embodiment, the connection destination of the cable 32 is controlled to be either one of the receiving antenna 30 and the terminal circuit 33 in a time period in which a wireless signal is received from the capsule endoscope 2, and a received strength of the wireless signal received by the receiving antenna 30 and cable 32 and a received strength of the wireless signal received by just the cable 32 are acquired. Thereafter, at the receiving unit 401, based on the received strengths that have been received, the received strength correcting unit 403 calculates a received strength of just the receiving antenna 30. This fourth embodiment enables accurate measurement of received strengths of a wireless signal received by the receiving antennas 30A to 30C.

The above described configurations and signal processing according to the second or third embodiment may be applied to this fourth embodiment.

Furthermore, an execution program for the processing executed by each component of the capsule endoscopes, receiving devices, and processing devices, of the capsule endoscope systems according to the first to fourth embodiments: may be configured to be provided by being recorded, in a file having an installable format or an executable format, on a computer readable recording medium, such as a CD-ROM, a flexible disk (FD), a CD-R, or a DVD; or may be configured to be provided by being stored on a computer connected to a network, such as the Internet, and being downloaded via the network. In addition, the execution program may be configured to be provided or distributed via a network, such as the internet.

Furthermore, according to the above description of the first to fourth embodiments, wireless signals are generated by the capsule endoscopes 2 that are radio wave transmitting devices, but the wireless signals are not necessarily generated by the capsule endoscopes 2 and may be generated by any device that generates and outputs wireless signals. For example, a pacemaker capable of being attached to a subject and generating and outputting a wireless signal may serve as a radio wave transmitting device.

The present disclosure has an effect of enabling accurate measurement of received strengths of wireless signals received by receiving antennas.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A receiving device comprising:
a receiving antenna configured to receive a wireless signal transmitted from a radio wave transmitting device;
a terminal circuit provided to the receiving antenna;
a cable configured to transmit the wireless signal, one end of the cable being connected to the receiving antenna or the terminal circuit;
a switch configured to switch, in a time period in which the wireless signal is transmitted, between a first connection state where the one end of the cable is connected to the receiving antenna and a second connection state where the one end of the cable is connected to the terminal circuit;
a measuring circuit configured to measures a first received strength of the wireless signal received by the receiving antenna and the cable in the first connection state and a second received strength of the wireless signal received by the cable in the second connection state; and
a calculator configured to calculate, based on the first and second received strengths, a third received strength of the wireless signal received by the receiving antenna.

2. The receiving device according to claim 1, wherein
the antenna includes a first receiving antenna and a second receiving antenna,
the terminal circuit includes a first terminal circuit and a second terminal circuit,
the cable includes a first cable and a second cable, and
the switch includes
a first switch configured to switch, in a first time period in the time period in which the wireless signal is transmitted, between the first connection state where one end of the first cable is connected to the first receiving antenna and the second connection state where the one end of the first cable is connected to the first terminal circuit, and
a second switch configured to switch, in a second time period, after the first time period, in the time period in which the wireless signal is transmitted the second time period, between the first connection state where one end of the second cable is connected to the second receiving antenna and the second connection state where the one end of the second cable is connected to the second terminal circuit.

3. The receiving device according to claim 1, wherein
the antenna includes a first receiving antenna and a second receiving antenna,
the terminal circuit includes a first terminal circuit and a second terminal circuit,
the cable includes a first cable and a second cable,
the switch includes
a first switch configured to switch, in the time period in which the wireless signal is transmitted, between the first connection state where one end of the first cable is connected to the first receiving antenna and the second connection state where the one end of the first cable is connected to the first terminal circuit, and
a second switch configured to switch, in the time period in which the wireless signal is transmitted the second time period, between the first connection state where one end of the second cable is connected to the second receiving antenna and the second connection state where the one end of the second cable is connected to the second terminal circuit, and
the first switch and the second switch are configured to switch the first and the second connection states in parallel to each other.

4. The receiving device according to claim 1, wherein
the radio wave transmitting device is a capsule endoscope,
the wireless signal includes a first information region related to pixels for generation of an image and a second information region related to information other than the pixels, and
the switch is configured to switch between the first connection state and the second connection state, in a time period in which a signal of the second information region is transmitted.

5. The receiving device according to claim 1, wherein
the radio wave transmitting device is a capsule endoscope,
the wireless signal includes: a first wireless signal including an image signal; and a second wireless signal for measurement of the first and second received strengths, and
the switch is configured to switch between the first connection state and the second connection state, in a time period in which the second wireless signal is transmitted.

6. A method of receiving a wireless signal from a radio wave transmitting device via a receiving antenna unit, the receiving antenna unit including: a receiving antenna configured to receive a wireless signal transmitted from a radio wave transmitting device; a terminal circuit provided to the receiving antenna; a cable configured to transmit the wireless signal, one end of the cable being connected to the receiving antenna or the terminal circuit; a switch configured to switch connection destinations of the one end of the cable, the method comprising:
switching to a first connection state where the one end of the cable is connected to the receiving antenna, in a time period in which the wireless signal is transmitted;
switching to a second connection state where the one end of the cable is connected to the terminal circuit;
measuring a first received strength of the wireless signal received by the receiving antenna and the cable in the first connection state and a second received strength of the wireless signal received by the cable in the second connection state; and
calculating, based on the first and second received strengths, a third received strength of the wireless signal received by the receiving antenna.
